(19) United States
(12) Patent Application Publication
(10) Pub. No.: US 2017/0153207 A1
(43) Pub. Date: Jun. 1, 2017

(54) ULTRASONIC SCANNER WITH A MAGNETIC COUPLING BETWEEN A MOTOR AND A MIRROR

(71) Applicant: Eye-Life AS, Narvik (NO)

(72) Inventor: Dr. Trygve Burchardt, Fredrikstad (NO)

(21) Appl. No.: 15/429,341

(22) Filed: Feb. 10, 2017

Related U.S. Application Data
(63) Continuation of application No. PCT/IB2015/056091, filed on Aug. 11, 2015.
(60) Provisional application No. 62/035,942, filed on Aug. 11, 2014.

(51) Int. Cl. G01N 29/265; G01N 29/24; A61B 8/00

(52) U.S. Cl. CPC G01N 29/265; A61B 8/4461; G01N 29/24; G01N 2291/26; G01N 2291/101

(57) ABSTRACT

An illustrative device for creating images via ultrasonic pulses comprises an electronics chamber and a probe head. The electronics chamber comprises a motor with an output shaft. The probe head is attached to the electronics chamber. The probe head includes a liquid-filled chamber that comprises an ultrasonic transducer configured to transmit and receive ultrasonic pulses and a mirror configured to reflect the ultrasonic pulses. The mirror is configured to rotate. The output shaft of the motor and the mirror are rotationally coupled.

### CROSS-REFERENCE TO RELATED APPLICATIONS

This application is a continuation of International Application PCT/IB2015/056091, filed Aug. 11, 2015, which claims priority to U.S. Provisional Patent Application Ser. No. 62/035,942 filed Aug. 11, 2014, both which are incorporated herein by reference in their respective entireties.

### FIELD

This disclosure relates to methods and apparatuses for imaging sections of a body by transmitting ultrasonic energy into the body and determining the characteristics of the ultrasonic energy reflected therefrom. More particularly, this disclosure relates to an improved ultrasonic scanning technique and system with a magnetic coupling between a motor and a reflector.

### BACKGROUND

The following description is provided to assist the understanding of the reader. None of the information provided or references cited is admitted to be prior art. Some ultrasonic imaging devices contain motors and liquid-filled chambers. However, inefficiencies and/or malfunctions can result from the use of motors in contact with the liquid in the liquid-filled chambers. Thus, it is desirable to improve efficiency and reduce malfunctions in ultrasound probes that have motors and liquid.

### SUMMARY

An illustrative device for creating images via ultrasonic pulses comprises an electronics chamber and a probe head. The electronics chamber comprises a motor with an output shaft. The probe head is attached to the electronics chamber. The probe head includes a liquid-filled chamber that comprises an ultrasonic transducer configured to transmit and receive ultrasonic pulses and a mirror configured to reflect the ultrasonic pulses. The mirror is configured to rotate. The output shaft of the motor and the mirror are rotationally coupled.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF DRAWINGS

FIG. 1A is a diagram of an ultrasound probe with a motor placed inside of a liquid-filled chamber from a top view in accordance with an illustrative embodiment.

FIG. 1B is a diagram of an ultrasound probe with a motor placed inside of a liquid-filled chamber from a side view in accordance with an illustrative embodiment.

FIG. 1C is diagram of a cross-sectional view of an ultrasound probe with a motor placed inside of a liquid-filled chamber in accordance with an illustrative embodiment.

FIG. 2A is a diagram of an ultrasound probe with a motor placed outside of a liquid-filled chamber with a motor shaft entering the liquid-filled chamber from a top view in accordance with an illustrative embodiment.

FIG. 2B is a diagram of an ultrasound probe with a motor placed outside of a liquid-filled chamber with a motor shaft entering the liquid-filled chamber from a side view in accordance with an illustrative embodiment.

FIG. 3A is a diagram of an ultrasound probe with a motor placed outside of a liquid-filled chamber utilizing a magnetic coupling from a top view in accordance with an illustrative embodiment.

FIG. 3B is a diagram of an ultrasound probe with a motor placed outside of a liquid-filled chamber utilizing a magnetic coupling from a side view in accordance with an illustrative embodiment.

FIG. 3C is a close-up illustration of the magnetic coupling in accordance with an illustrative embodiment.

FIGS. 4A-4D are illustrations of various configurations of magnets in accordance with illustrative embodiments.

FIG. 5 illustrates a configuration of magnets in the magnetic coupling in accordance with an illustrative embodiment.

FIG. 6A is a diagram of an ultrasound probe with a motor placed outside of a liquid-filled chamber utilizing a magnetic coupling and a detachable head from a top view in accordance with an illustrative embodiment.

FIG. 6B is a diagram of an ultrasound probe with a motor placed outside of a liquid-filled chamber utilizing a magnetic coupling and a detachable head from a side view in accordance with an illustrative embodiment.

FIG. 6C is a close-up illustration of the magnetic coupling with a detachable head in accordance with an illustrative embodiment.

FIGS. 6D-6F are diagrams of the ultrasound probe illustrated in FIGS. 6A-6C, respectively, that show the rotation of the motor axis and the mirror axis.

FIG. 7A is a cross-sectional illustration of a magnetic coupling with multiple magnets on each shaft in accordance with an illustrative embodiment.

FIG. 7B is an illustration of multiple magnets on a shaft in accordance with an illustrative embodiment.

FIG. 8 is a diagram illustrating an orientation of the magnetic coupling with the motor shaft and the mirror shaft perpendicular to one another in accordance with an illustrative embodiment.

FIG. 9 is a diagram illustrating an orientation of the magnetic coupling with the motor shaft and the mirror shaft perpendicular to one another using an L-coupling in accordance with an illustrative embodiment.

FIGS. 10A-10E are illustrations of an ultrasound probe with a removable head from an outside perspective in accordance with an illustrative embodiment.

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Ultrasound imaging techniques are often used in clinical diagnostics. Ultrasound differs from other forms of radiation used for imaging in its interaction with living systems in that ultrasound is a mechanical wave. Accordingly, the information provided by the use of ultrasonic waves is of a different nature than that obtained by other methods and is found to be complementary to other diagnostic methods, such as those employing X-rays. Also, the risk of tissue damage using ultrasound appears to be much less than the apparent risk associated with ionizing radiations such as X-rays. Ultrasound imaging devices can be used in settings other than a medical setting. For example, ultrasound imaging devices can be used for finding cracks in materials (e.g., metals, steel, etc.), diagnosing machinery malfunctions, etc.

Many diagnostic techniques using ultrasound are based on a pulse-echo method wherein pulses of ultrasonic energy are periodically generated by a suitable piezoelectric transducer. Each short pulse of ultrasonic energy is focused to a narrow beam, which is transmitted into the patient's body in which the energy eventually encounters interfaces. Once the energy encounters interference, a portion of the ultrasonic energy is reflected at the boundary back to the transducer. After generation of the pulse, the transducer operates in a “listening” mode in which the device converts received reflected energy, or “echoes,” from the body into electrical signals. The time of arrival of the echoes depends on the distance of the interfaces from the device and the propagation velocity of the ultrasonic energy. The amplitude of the echoes is indicative of the reflection properties of the interphase and, accordingly, of the nature of the characteristic structure forming the interphase. In alternative embodiments, any suitable method of transmitting and/or receiving ultrasonic energy may be used.

There are various ways in which the information in the received echoes can be usefully presented. One common form of display is referred to as a “B-scan.” In a B-scan, the echo information is of a form similar to a conventional television display. That is, the received echo signals are utilized to modulate the brightness of the display at each point scanned. This type of display is useful, for example, when the ultrasonic energy is scanned transverse to the body so that individual “ranging” information yields individual scan lines on the display, and successive transverse positions are utilized to obtain successive scan lines on the display. The two-dimensional B-scan technique yields a cross-sectional picture in the plane of the scan, and the resultant display can be viewed directly and/or be recorded. In most instances, ultrasonic energy is almost totally reflective at interfaces with gas. Thus, coupling fluid, such as water or oil, or a direct-contact type transducer can be used to limit the amount of gas through which the ultrasonic energy passes.

An illustrative type of apparatus having a console includes a timing signal generator, energizing and receiving circuitry, and a display/recorder for displaying and/or recording image-representative electronic signals, such as those described in U.S. Pat. No. 4,084,582 and U.S. Pat. No. 6,712,765, which are incorporated herein by reference in their entirety. A portable scanning head or module, suitable for being hand held, can have a fluid-tight enclosure having a scanning window formed of a flexible material. A transducer in the portable scanning module converts energy and also converts received ultrasound echoes into electrical signals, which are coupled to the receiver circuitry. A focusing lens is coupled to the transducer, and a fluid, such as water or oil, fills the portable scanning module in the region between the focusing lens and the scanning window. A reflective scanning mirror is disposed in the fluid, and a driving motor, energized in synchronism with the timing signals, drives the scanning mirror in a periodic fashion. The ultrasonic beam is reflected off of the scanning mirror and into the body being examined via a scanning window formed of a rigid material.

For a two dimensional B-scan taken with the above-described type of scanning head, the dimensions scanned are: (1) depth into the body, which varies during each display scan line by virtue of the ultrasonic beam travelling deeper into the body as time passes; and (2) a slower transverse scan caused by the scanning mirror. The display is typically in a rectangular format (for example, the familiar television type of display with linear sweeps in both directions). However, the transverse scan of the ultrasonic beam itself, as implemented by the scanning mirror, results in a sector scan. For distances deeper in the body, the fanning out of the sectors results in geometrical distortion when displayed on a linear rectangular display. For example, the azimuth dimension in the extreme far field may be, for example, 2.5 times larger than the azimuth dimension in the extreme near field. Thus, the density of information on the far field side of the display is much higher than the density of information on the near field side of the display. In other words, what appears to be equal distance in the body on the far field side and the near field side of the display are actually different distances.

In some embodiments, the scanning window is in the form of an acoustic lens for converging the scan of the ultrasonic beam incident thereon that forms within the enclosure, as in U.S. Pat. No. 4,325,381, which is incorporated herein by reference in its entirety. In some embodiments, the acoustic lens reduces geometric distortion of the scan of the ultrasonic beam. In an embodiment, the window/lens is formed of a rigid plastic material in a substantially plano-concave shape, with the concave surface facing the outside of the enclosure. In such an embodiment, the window/lens is provided with a focal length of about 1.5 times the distance between the reflective scanning means and the windows/lens and may be particularly suitable for a functioning embodiment. In alternative embodiments, any suitable lens, window, focal length, etc. may be used.

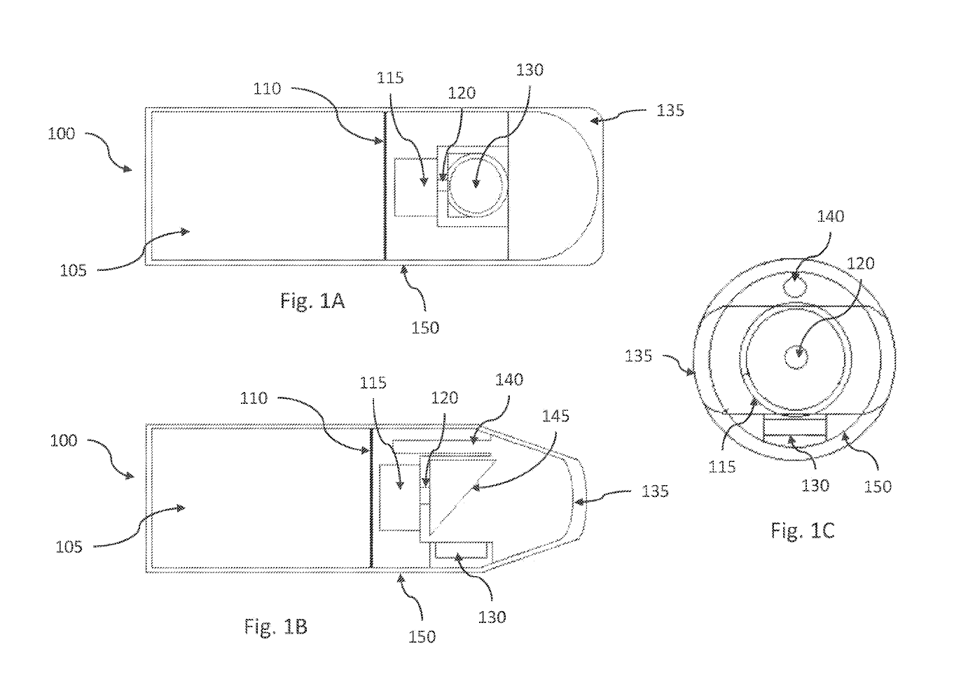

FIG. 1A is a diagram of an ultrasound probe with a motor placed inside of a liquid-filled chamber from a top view in accordance with an illustrative embodiment. FIG. 1B is a diagram of an ultrasound probe with a motor placed inside of a liquid-filled chamber from a side view in accordance with an illustrative embodiment. FIG. 1C is diagram of a cross-sectional view of an ultrasound probe with a motor placed inside of a liquid-filled chamber in accordance with an illustrative embodiment. In alternative embodiments, additional, fewer, and/or different elements may be used. An ultrasound probe 100 includes an electronics chamber 105, a wall 110, a motor 115, a motor shaft 120, a transducer 130, a lens 135, a cavity 140, a mirror 145, and a probe head 150.

The electronics chamber 105 houses electronics and possibly batteries and is attached to the probe head 150. In some embodiments, the chamber 105 is removably attached to the probe head 150. The probe head 150 includes the motor 115 that is attached to the mirror 145. The angle of the mirror 145 and the location of the transducer 130 are arranged such that the propagation pathway of the ultrasonic pulses is through the lens 135, is reflected by the mirror 145, and is received/transmitted by the transducer 130. In the embodiment illustrated in FIGS. 1A and 1B, the plane of the lens 135 is perpendicular to the transducer 130 and the angle of the mirror is 45°. In other embodiments, alternative configurations for the plan of the lens 135 and the angle of the mirror 145 may be used.

The probe head 150 (including cavity 140) can be liquid filled. The liquid can be any suitable liquid such as water or oil. The cavity 140 is positioned on the opposite side of the mirror 145 from the transducer 130.

As the motor 115 spins, the mirror 145 spins, thereby altering the path of the ultrasonic pulses emitted by the transducer 130 and transmitted through the lens 135. The altered path allows the ultrasound probe 100 to scan the medium at the end of the ultrasound probe 100 (e.g., a human body). Prolonged rotation of the motor 115 can lead to wear and tear of the bearings and gaskets in the motor 115.

Because the probe head 150 is liquid filled, the available motors 115 are limited to the types of motors that can operate while immersed in the liquid. However, even motors 115 that are suited to operating in liquid have a relatively short life span because prolonged operations can cause corrosion of the gaskets and seals caused by the rotating shaft. Leaks into the motor 115 can cause the motor 115 to lose efficiency and/or malfunction. Leaks into the motor 115 can also cause air to escape from the motor 115 into the liquid-filled chamber. If the probe head 150 has air in the liquid-filled chamber (e.g., cavity 140), the ultrasound probe 100 can produce inaccurate readings, signal noise, and/or reduced quality of the ultrasound image.

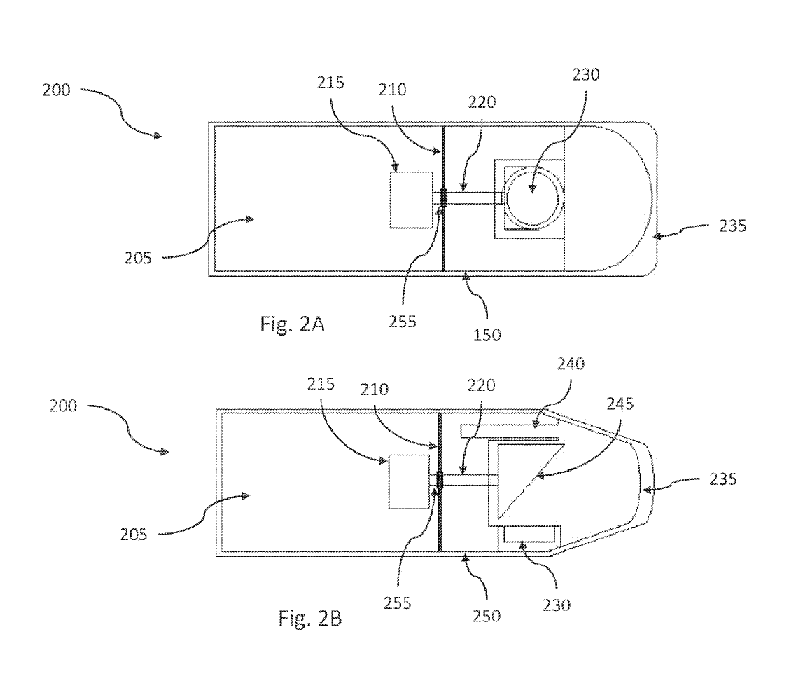

FIG. 2A is a diagram of an ultrasound probe with a motor placed outside of a liquid-filled chamber with a motor shaft entering the liquid-filled chamber from a top view in accordance with an illustrative embodiment. FIG. 2B is a diagram of an ultrasound probe with a motor placed outside of a liquid-filled chamber with a motor shaft entering the liquid-filled chamber from a side view in accordance with an illustrative embodiment. In alternative embodiments, additional, fewer, and/or different elements may be used. An ultrasound probe 200 includes an electronics chamber 205, a wall 210, a motor 215, a motor shaft 220, a transducer 230, a lens 235, a cavity 240, a mirror 245, a probe head 250, and a seal 255.

The various elements of ultrasound probe 200 operate and function similar to the ultrasound probe 100 of FIGS. 1A and 1B, except that the motor 215 is located within the electronics chamber 205. Accordingly, the shaft 220 passes through a hole in the wall 210. Electrical connections can pass through the wall 210 using any suitable means. The electrical connections can include connections between the transducer 230 and electronics and/or batteries housed in the electronics chamber 205. A seal 255, which may include a gasket, o-ring, or any other suitable device, is used to prevent the liquid from the liquid-filled chamber from leaking into the electronics chamber 205. By having the motor 215 located in the electronics chamber, which is not liquid filled, the amount of sealing necessary to protect the motor 215 from the liquid is reduced. For example, instead of sealing the entire motor 215 to prevent liquid from entering the motor 215, a seal 255 (which may contain one or more o-rings, gaskets, etc.) is used around the motor shaft 220. Reducing the amount or number of seals used to protect the motor 215 from liquid reduces the number of potential failure points. Additionally, in some embodiments, if the seal 225 fails, the liquid may leak into the electronics chamber 205, but not necessarily into the motor 215.

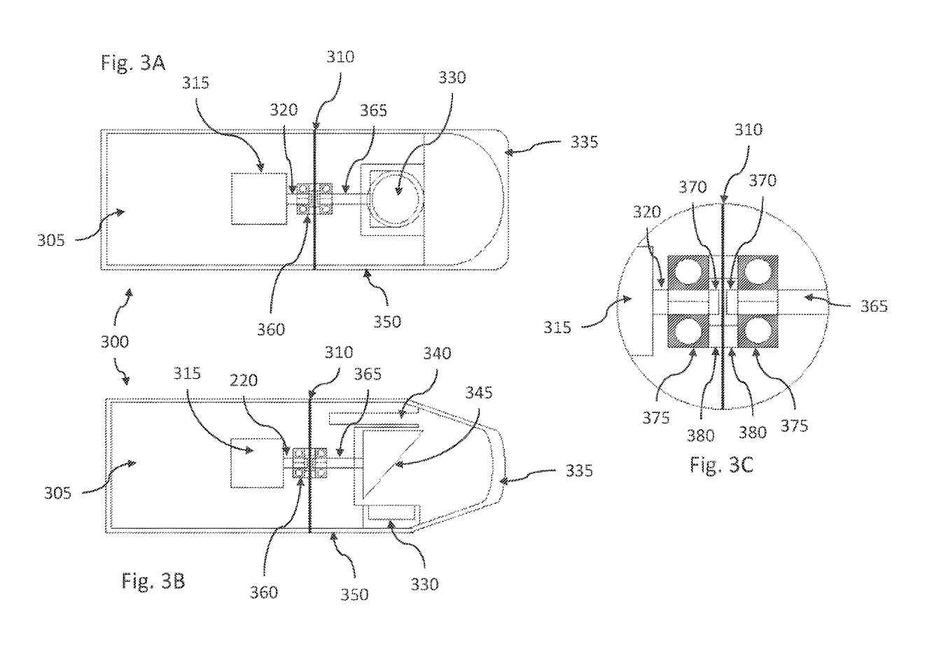

FIG. 3A is a diagram of an ultrasound probe with a motor placed outside of a liquid-filled chamber utilizing a magnetic coupling between the motor and a mirror shaft from a top view in accordance with an illustrative embodiment. FIG. 3B is a diagram of an ultrasound probe utilizing a motor placed outside of a liquid-filled chamber with a magnetic coupling between the motor and a mirror shaft from a side view in accordance with an illustrative embodiment. FIG. 3C is a close-up illustration of the magnetic coupling in accordance with an illustrative embodiment. In alternative embodiments, additional, fewer, and/or different elements may be used. An ultrasound probe 300 includes an electronics chamber 305, a wall 310, a motor 315, a motor shaft 320, a transducer 330, a lens 335, a cavity 340, a mirror 345, a probe head 350, a magnetic coupler 360, a mirror shaft 365, magnets 370, bearings 375, and a spacer 380.

The various elements of ultrasound probe 300 are similar to the elements of the ultrasound probe 200 of FIGS. 2A and 2B, except that the motor 315 is coupled through the wall 310 to a mirror shaft 365 via a magnetic coupling 360. Thus, in such an embodiment, there is no physical, mechanical connection between the motor 315 and the mirror 330. However, the motor shaft 315 and the mirror shaft 365 are rotationally coupled in a fashion such that the motor shaft 315 and the mirror shaft 365 rotate in concert with one another. The magnetic fields of the magnets 370 of the magnetic coupling 360 can pass through the wall 310. Thus, the wall 310 does not require a hole through which a shaft (e.g., motor shaft 220) passes to mechanically connect the mirror 345 with the motor 315. In some embodiments, the fluid-filled chamber of the ultrasound probe 300 is completely sealed. Accordingly, there is no risk that the motor 315 will wear out seals, gaskets, etc., used to separate the motor 315 from liquid in the liquid-filled chamber of the ultrasound probe 300. Thus, there is no risk that the seals, gaskets, etc. will lead to premature failure of the motor 315 as is possible in the ultrasound probe 100 and the ultrasound probe 200.

The magnetic coupling 360 includes ball bearings 375 on both sides of the wall 310. The ball bearings 375 can align the motor shaft 320 and the mirror shaft 365 such that the center axes of the motor shaft 320 and the mirror shaft 365 are aligned. The spacers 380 are fastened to the ball bearings 375 and the wall 310. The magnets 370 are attached to the ends of the motor shaft 320 and the mirror shaft 365. As shown in FIG. 3C, spacers 380 can be used such that the ball bearings 375 provide the magnets 370 with enough room to rotate freely. That is, the spacers 380 space the ball bearings 375 away from the wall 310 such that the magnets 370 do not touch the wall 310 or the bearings 375. When the magnets 370 rotate against the wall 310, friction can cause erosion of the wall 310 and/or the magnets 370.

In an illustrative embodiment, the magnets 370 are (about) 1 millimeter (mm) thick, as measured from the tip of the motor shaft 220. In alternative embodiments, the magnets 370 can be thinner or thicker than 1 mm. For example, the magnets 370 can be 0.5 mm, 0.75 mm, 0.9 mm, 0.95 mm, 1 mm, 1.05 mm, 1.1 mm, 1.25 mm, 1.5 mm, etc. thick. In an illustrative embodiment, the spacers 380 are less than 3 mm thick. For example, the spacers 380 can be 1.5 mm, 2 mm, 2.5 mm, 3 mm, etc. thick. In alternative embodiments, the spacers 380 can be more than 3 mm thick. The thickness of the spacers can depend on the strength and the number of magnets used. For example, the more magnetics used and/or the higher the strength of the magnets can allow the spacers to be thicker because the magnets can be moved further away from the wall 310 and still be effectively coupled.

In an illustrative embodiment, the wall 310 is less than 5 mm thick. For example, the wall 310 can be 3 mm thick. In other embodiments, the wall can be 4 mm, 3.5 mm, 2.5 mm, 2 mm, etc. thick. In an illustrative embodiment, the ball bearings have a diameter of between 5 mm and 10 mm. For example, the diameter of the ball bearings can be 5 mm, 6 mm, 7 mm, 8 mm, 9 mm, 10 mm, etc. In alternative embodiments, the diameter of the ball bearings can be less than 5 mm or greater than 10 mm. In some embodiments, the thickness of the motor shaft 220 (and the various other shafts described herein, such as the mirror shaft 365 and the L-coupler shaft 998) is about 5 mm in diameter. In alternative embodiments, the diameter of the motor shaft 220 is greater than or less than 5 mm. For example, the diameter of the motor shaft 220 can be 3 mm, 4 mm, 4.5 mm, 5.5 mm, 6 mm, 7 mm, etc.

In the embodiment shown in FIG. 3C, the magnets 370 are the same diameter as the motor shaft 320 and the mirror shaft 365. In alternative embodiments, different diameters can be used. For example, in some embodiments, the magnets 370 can be larger or smaller than the diameter of the motor shaft 320 and the mirror shaft 365. In other embodiments, the motor shaft 320 can be a different size than the mirror shaft 365. In yet other embodiments, the magnets 370 can be different sizes. In the embodiment illustrated in FIGS. 3A-3C, the magnets 370 are centered along the rotational axes of the motor shaft 320 and the mirror shaft 365.

As in the embodiment illustrated in FIG. 3C, spacers 380 can be used to place the ball bearings 375 in a position such that the ball bearings 375 contact the motor shaft 320 and the mirror shaft 365 and do not contact the magnets 370. As noted above, in some embodiments, the magnets 370 can be larger than the motor shaft 320 and the mirror shaft 365. In such embodiments, the spacers 380 can be positioned to allow the magnets 370 to rotate without interference from the bearings 375. In embodiments in which the magnets 370 are not larger than the motor shaft 320, spacers 380 can be positioned such that the ball bearings 375 do not contact (e.g., ride on) the magnets 370. Using the spacers 380 can reduce the mechanical forces (e.g., vibration, friction, etc.) on the magnets 370, thereby reducing degradation of the magnets 370 and prolonging the useful life of the magnets 370. In some embodiments, ball bearings 375 may not be used. In some embodiments, any other suitable means for aligning the magnets 370 and/or reducing rotational friction can be used.

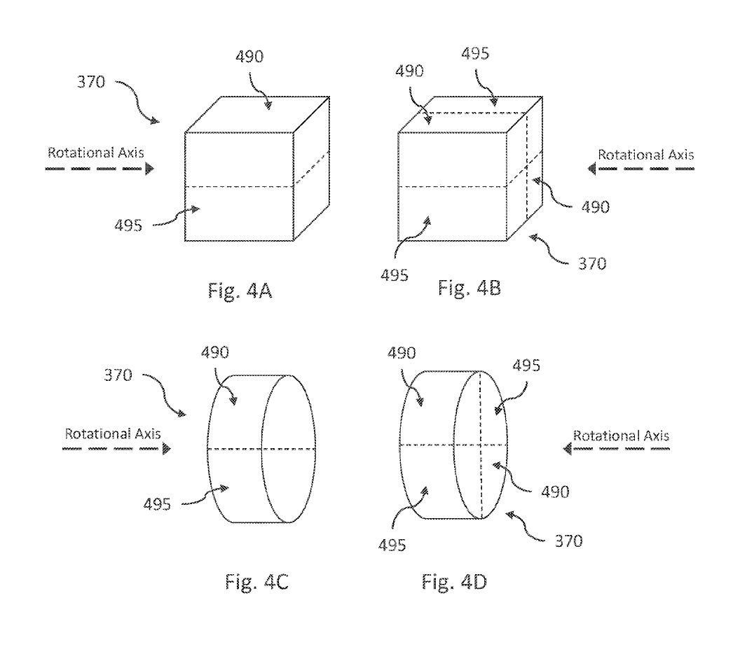

The magnetic coupling 360 allows the motor 315 to drive the mirror 345 without having the motor shaft 320 physically contact the mirror shaft 365. The magnets 370 can be configured in any suitable manner to couple the rotation of the motor shaft 320 to the rotation of the mirror shaft 365. FIGS. 4A-4C are illustrations of various configurations of magnets 370 in accordance with illustrative embodiments. In alternative embodiments, additional, fewer, and/or different elements may be used. Also, the illustration of magnets 370 is meant to be illustrative only and is not meant to be limiting with regard to proportion or shape. Each of FIGS. 4A-4C illustrates one magnet 370. Accordingly, in some embodiments, the motor shaft 320 and the mirror shaft 365 each have a magnet 370. In some embodiments, the magnets 370 on the motor shaft 320 and the mirror shaft 365 have the same shape and configuration. In alternative embodiments, the magnets 370 on the motor shaft 320 and the mirror shaft 365 have a different shape and/or configuration.

As shown in FIGS. 4A and 4B, the magnet 370 can be a hexahedron (e.g., a cube, a rectangular cuboid, etc.). As shown in FIGS. 4C and 4D, the magnet 370 can be a cylinder (e.g., a circular cylinder, an elliptic cylinder, etc.). In some instances, the shape of the magnets 370 are chosen based on mechanical design features (e.g., clearance, spacing, etc.). In the embodiments illustrated in FIGS. 4A and 4C, the magnet 370 comprises one magnet (e.g., two poles). The magnet can comprise a north pole 490 and a south pole 495. In alternative embodiments, such as those illustrated in FIGS. 4B and 4D, the magnet 370 can comprise multiple magnets with north poles 490 and south poles 495. The distinction between the north poles 490 and the south poles 495 of FIGS. 4A-4D are illustrated using dashed lines.

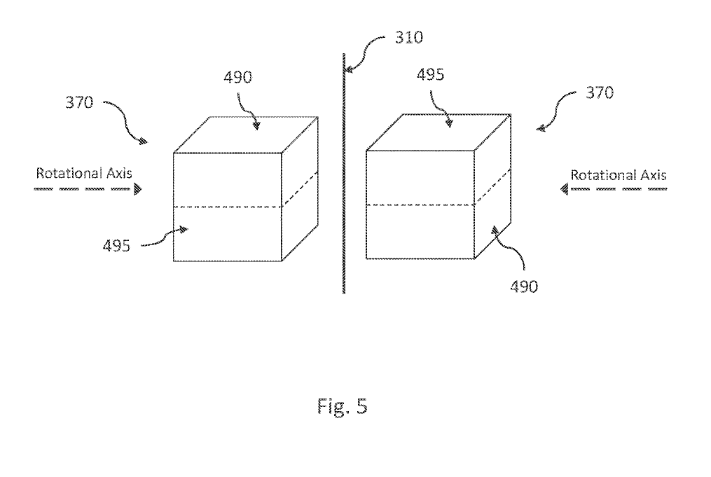

FIG. 5 illustrates a configuration of magnets in the magnetic coupling in accordance with an illustrative embodiment. In alternative embodiments, additional, fewer, and/or different elements may be used. Because the magnet 370 associated with the mirror is free to rotate in either rotational direction, the magnetism of the magnets 370 causes the magnets 370 to orient themselves such that opposing poles of the magnets 370 are closest to one another, as illustrated in FIG. 5. Accordingly, in some embodiments, when the magnets 370 are brought within operating distances of one another (e.g., by attaching the probe head 350 to the electronics chamber 305), the magnets 370 will automatically rotate to orient themselves to the orientation illustrated in FIG. 5. Thus, in some embodiments, the motor 315 and the mirror 345 are automatically oriented to one another in the same position each time the probe head 350 and the electronics chamber 305 are connected.

Although magnets 370 of FIG. 4A are illustrated in FIG. 5, any suitable magnets 370 may be used. Each of the magnets 370 can be attached to a motor shaft 320 or a mirror shaft 365. As in the embodiment illustrated in FIGS. 3A-3C, the magnets 370 can be separated by a wall 310. The wall 310 can be comprised of any suitable material that will allow the magnetic fields of the magnets 370 to interact with one another. The rotational axes of the magnets 370 is illustrated in FIG. 5. The magnet 370 that is attached to the mirror shaft 365 can be configured to rotate with minimal rotational friction. Accordingly, the magnetic forces of the magnets 370 will rotate the magnets 370 into a position in which the north pole 490 of one magnet 370 is closest to the south pole 490 of the other magnet 370. As the magnet 370 attached to the motor shaft 320 rotates, the magnet 370 attached to the mirror shaft 365 will rotate with the magnet 370 attached to the motor shaft 320 to maintain the alignment illustrated in FIG. 5.

The inventors have designed, built, and tested an ultrasound probe with a magnetic coupling as illustrated in FIGS. 3A-3C. The motor speed was pre-programmed to spin at various settings (e.g., settings 1-6). In general, the various speed settings are (approximate) multiples of the first speed setting. The use of these speed settings shows that, given a setpoint (which can be arbitrary), the mirror will spin at the same speed consistently and reliably, regardless of the speed setting. Ten measurements were taken of the rotational speed of the mirror for each of the six pre-programmed motor settings. Each measurement was the average revolutions per minute (rpm) of the mirror over a thirty-second recording time (which included about 5-10 readings per measurement). The results in rpm of the tests are shown in the table below:

| | Motor | Motor | Motor | Motor | Motor | Motor |
|---|---|---|---|---|---|---|
| Measurement | Setting | Setting | Setting | Setting | Setting | Setting |
| Number | 1 | 2 | 3 | 4 | 5 | 6 |
| 1 | 104 | 214 | 322 | 430 | 538 | 648 |
| 2 | 104 | 215 | 324 | 428 | 537 | 640 |
| 3 | 103 | 214 | 322 | 430 | 540 | 649 |
| 4 | 103 | 214 | 322 | 428 | 540 | 648 |
| 5 | 103 | 214 | 324 | 431 | 542 | 648 |
| 6 | 104 | 213 | 324 | 432 | 540 | 646 |
| 7 | 103 | 213 | 324 | 430 | 537 | 648 |
| 8 | 103 | 214 | 322 | 428 | 540 | 648 |
| 9 | 104 | 214 | 324 | 432 | 544 | 647 |
| 10 | 101 | 214 | 324 | 430 | 539 | 648 |

As illustrated in the table, the rotational speed of the motor was matched by the rotational speed of the mirror through the magnetic coupling in a reliable, accurate, and consistent manner.

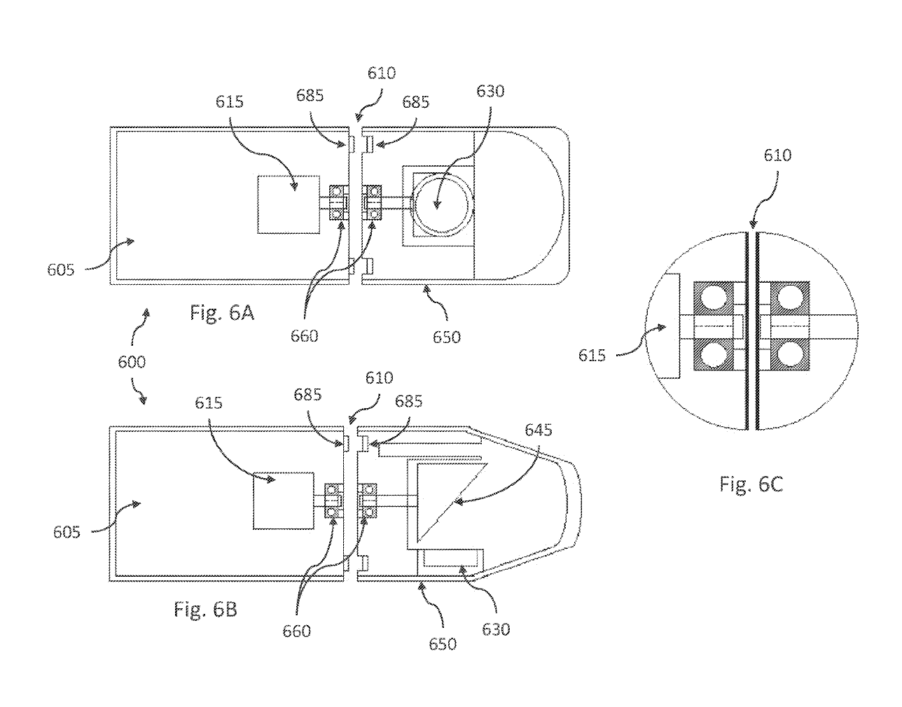

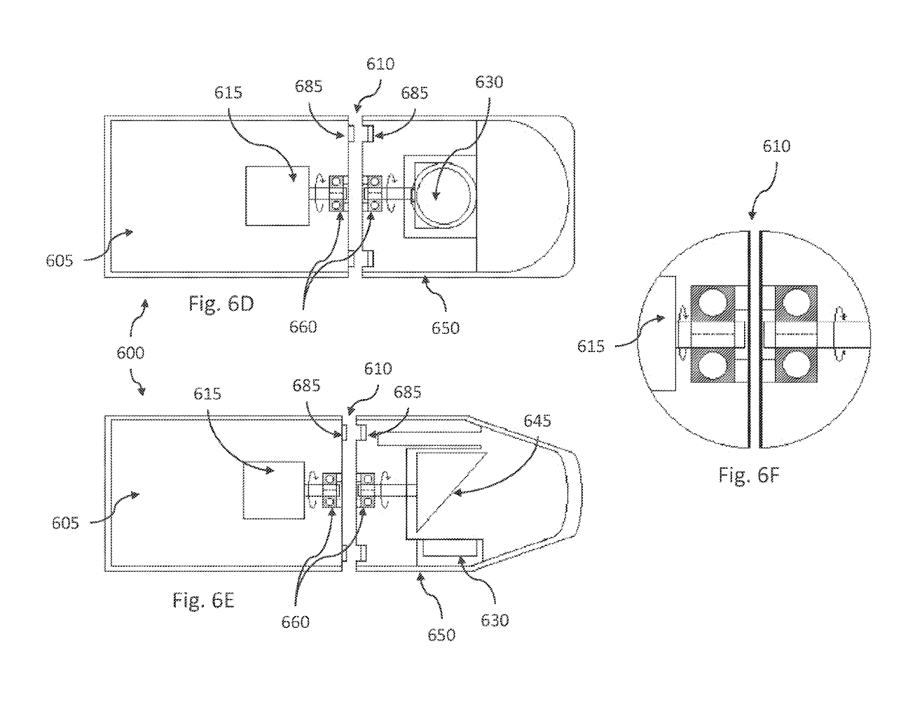

FIG. 6A is a diagram of an ultrasound probe with a motor placed outside of a liquid-filled chamber utilizing a magnetic coupling and a detachable head from a top view in accordance with an illustrative embodiment. FIG. 6B is a diagram of an ultrasound probe with a motor placed outside of a liquid-filled chamber utilizing a magnetic coupling and a detachable head from a side view in accordance with an illustrative embodiment. FIG. 6C is a close-up illustration of the magnetic coupling with a detachable head in accordance with an illustrative embodiment. In alternative embodiments, additional, fewer, and/or different elements may be used. An ultrasound probe 600 includes an electronics chamber 605, a wall 610, a motor 615, a transducer 630, a mirror 645, a probe head 650, and a magnetic coupler 660, and alignment means 685.

The ultrasound probe 600 has similar elements as the ultrasound probe 300 of FIGS. 3A-3C, except that the probe head 650 is detachable from the electronics chamber 605. In this way, multiple different probe heads may be interchangeably used with the electronics chamber 605. Thus, the wall 610 comprises a wall on the electronics chamber and a wall on the probe head 650. The alignment means 685 can be used to align the probe head 650 with the electronics chamber 605 such that the rotational axes of each end of the magnetic coupling 660 are aligned. Magnets within the tongue and groove alignment means 685 can be used to removably fix the probe head 650 to the electronics chamber 605. Although the alignment means 685 illustrated in FIGS. 6A and 6B are a tongue and groove arrangement, any suitable means for aligning the probe head 650 with the electronics chamber 605 can be used. FIGS. 6A-6C show the ultrasound probe 600 with the probe head 650 detached from the electronics chamber 605, as illustrated by the gap between the walls 610. Attachment of the probe head 650 to the electronics chamber 605 can be accomplished by eliminating the gap between the walls 610 by pressing the wall of the probe head 650 against the wall of the electronics chamber 605. In some embodiments, the electronics chamber 605 and/or the probe head 650 of one ultrasound probe 600 can be interchanged with the electronics chamber 605 and/or the probe head 650 of another.

FIGS. 6D-6F are diagrams of the ultrasound probe illustrated in FIGS. 6A-6C, respectively, that show the rotation of the motor axis and the mirror axis. Although FIGS. 6A-6C illustrate the motor axis and the mirror axis spinning in one direction, in alternative embodiments, the axes can spin in the opposite direction.

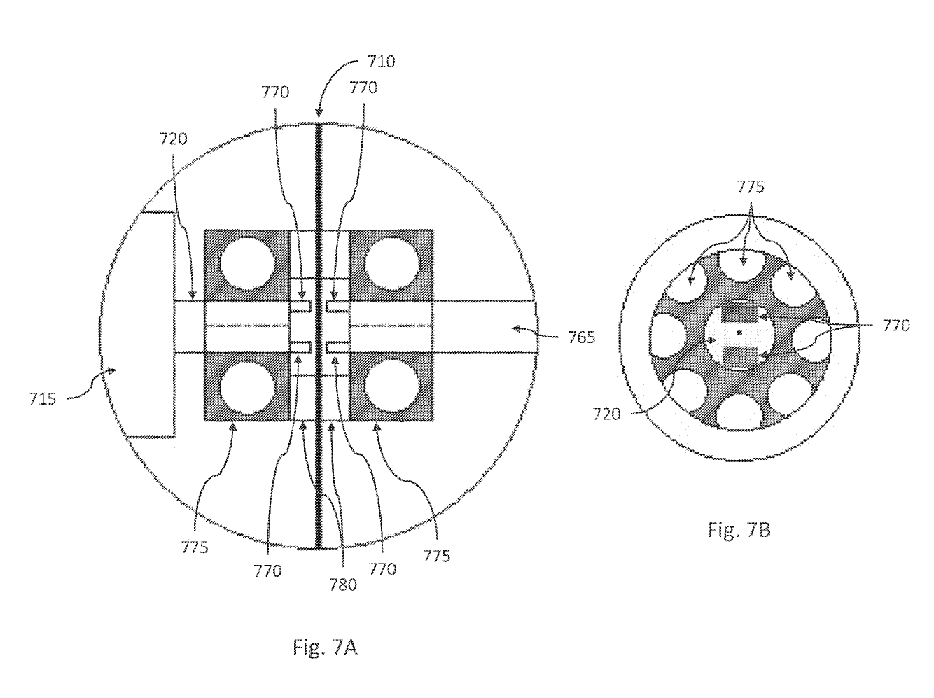

FIG. 7A is a close-up cross-sectional illustration of a magnetic coupling with multiple magnets on each shaft in accordance with an illustrative embodiment. In alternative embodiments, additional, fewer, and/or different elements may be used. An illustrative ultrasound probe has a wall 710, a motor 715, a motor shaft 720, a mirror shaft 765, magnets 770, ball bearings 775, and spacers 780. The various elements of FIG. 7A are similar to the elements shown in FIG. 3C, except that the motor shaft 720 and the mirror shaft 765 each have multiple magnets 770. The use of multiple magnets allows for positioning of the mirror. The magnets 770 illustrated in 7A are arranged with the poles of each magnet 770 aligning along the axial length of the motor shaft 720 and the mirror shaft 765. That is, one magnet 770 has two poles: one on the mirror side and one on the motor side. In such an embodiment, as shown in FIG. 7A, the motor shaft 720 and the mirror shaft 765 each have two magnets 770. The magnets 770 of the respective shaft are arranged such that the poles are in opposite directions. For example, one of the magnets 770 of the motor shaft 720 has the north pole towards the mirror side, and the other magnet 770 of the motor shaft 720 has the south pole towards the mirror side. In an illustrative embodiment, the elements marked by reference number 770 in FIG. 7A are poles of a magnet and not individual magnets (each with two poles).

FIG. 7B is an illustration of multiple magnets on a shaft in accordance with an illustrative embodiment. In alternative embodiments, additional, fewer, and/or different elements may be used. Attached to the motor shaft 720 (which, in FIG. 7B, can also be used to illustrate the mirror shaft 765) are magnets 770. In alternative embodiments, the elements marked by reference number 770 are poles of a magnet. The motor shaft 770 is surrounded by ball bearings 775. Although FIG. 7B does not illustrate the ball bearings 775 contacting the motor shaft 770, in alternative embodiments, at least one of the ball bearings 775 contacts the motor shaft 770.

When the probe head (e.g., probe head 650) is disconnected from the electronics chamber (e.g., electronics chamber 605) and then re-connected, the magnets 770 of the mirror shaft 765 will automatically align themselves to the magnets 770 of the motor shaft 720. The automatic alignment reduces the need for calibration of the mirror position before using the device after re-connection of the probe head and electronics chamber. The use of magnets 770 can also be used to fix a zero point position on the mirror for calibration of the ultrasound probe such that the ultrasonic pulses are periodically fired when the mirror is in the correct position.

In alternative embodiments, the orientation of the motor shaft and the mirror shaft may not be in line with one another. For example, the motor shaft and the mirror shaft may be perpendicular to one another. In some instances, having the shafts perpendicular to one another allows greater flexibility in the design of the probe head. In addition, having the mirror rotate about an axis that is perpendicular to a transmitting surface of the transducer can make aligning the transducer with the mirror easier and more reliable.

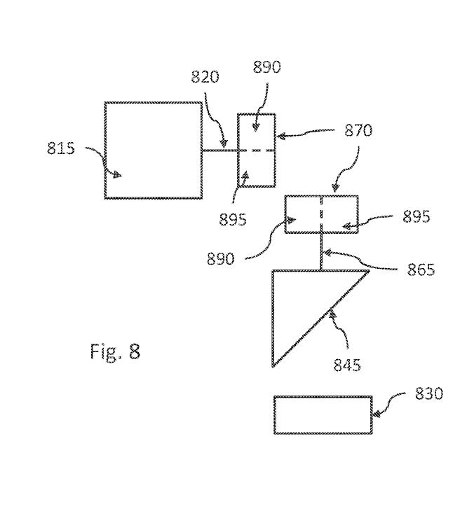

FIG. 8 is a diagram illustrating an orientation of the magnetic coupling with the motor shaft and the mirror shaft perpendicular to one another in accordance with an illustrative embodiment. In alternative embodiments, additional, fewer, and/or different elements may be used. FIG. 8 is meant to be illustrative only and is not meant to be limiting with respect to size, relational position, scale, etc. In the embodiment illustrated in FIG. 8, a motor 815 is attached to a magnet 870 via a motor shaft 820. A mirror 845 is attached to a magnet 870 via a mirror shaft 865. The magnets 870 have north poles 890 and south poles 895. As the motor 815 rotates the corresponding magnet 870, the magnet 870 corresponding to the mirror 845 also rotates, thereby rotating the mirror 845. A transducer 830 is at an angle to the reflective surface of the mirror 845 suitable to reflect ultrasonic signals out of the front end of the probe head (not illustrated in FIG. 8).

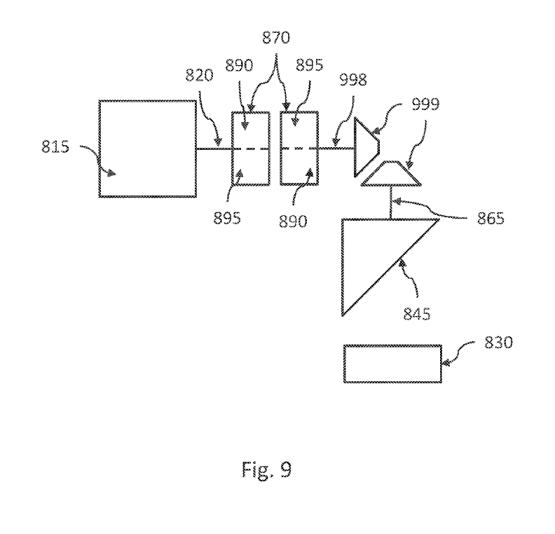

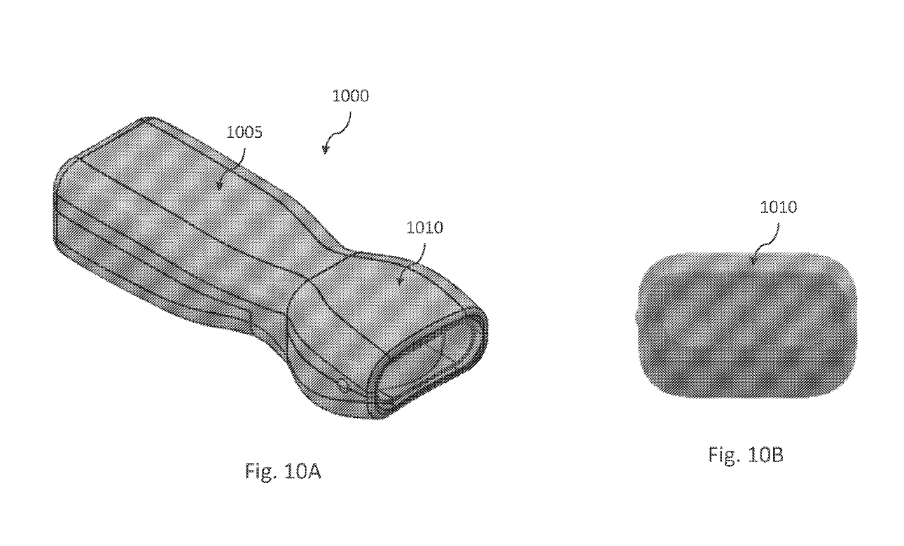

FIG. 9 is a diagram illustrating an orientation of the magnetic coupling with the motor shaft and the mirror shaft perpendicular to one another using an L-coupling in accordance with an illustrative embodiment. In alternative embodiments, additional, fewer, and/or different elements may be used. FIG. 9 is meant to be illustrative only and is not meant to be limiting with respect to size, relational position, scale, etc. For example, although the gears 999 are not illustrated as touching in FIG. 9, it is to be understood that the gears 999 engage one another.

The elements of FIG. 9 are similar to the elements of FIG. 8 except that the orientation of the magnets is linear, as in embodiments illustrated in FIGS. 3A-3B. But the mirror shaft 865 is perpendicular to the motor shaft 820, as in the embodiment illustrated in FIG. 8. The magnet 870 associated with the mirror 845 is attached to an L-coupler (with gears 999) via an L-coupler shaft 998. The L-coupler comprises gears 999 that translate rotational energy along the L-coupler shaft 998 into rotational energy along the mirror shaft 865, which is perpendicular to the L-coupler shaft 998. The gears 999 can be, for example, bevel gears. Although a 90° L-coupler is illustrated in FIG. 9, any other suitable device for transferring rotational motion to another axis may be used. Thus, when the motor 815 rotates the corresponding magnet 870 via the motor shaft 820, the magnet 870 corresponding to the mirror 845 rotates, and the gears 999 of the L-coupler transfer rotational energy of the L-coupler shaft 998 into rotational energy of the mirror shaft 865.

In some instances, the linear alignment of the magnets 870, as opposed to the perpendicular alignment of magnets 870 in FIG. 8, increases the reliability and stability of the magnetic coupling while maintaining the flexibility in the design of the probe head and increased ease of aligning the transducer 830 with the mirror 845.

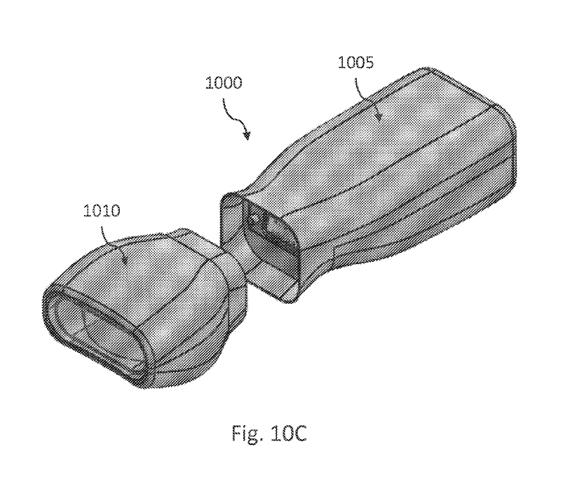

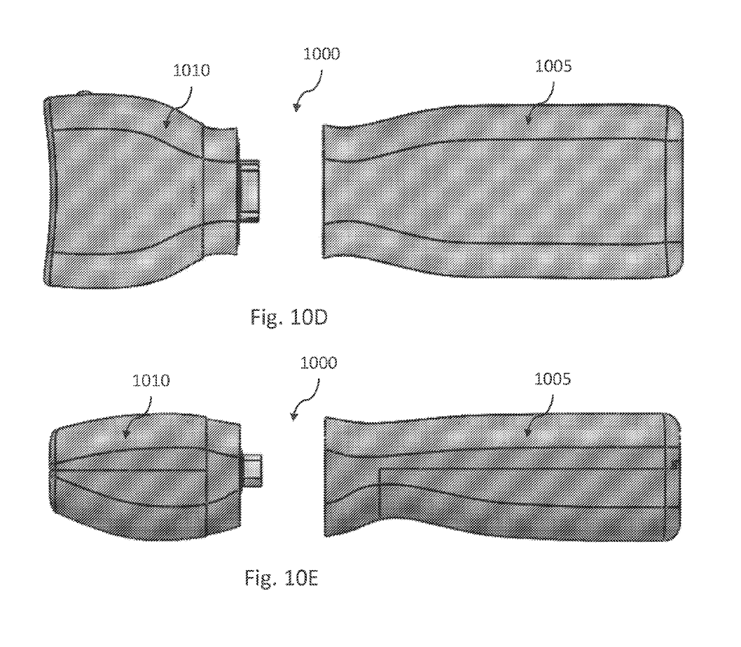

FIGS. 10A-10E are illustrations of an ultrasound probe with a removable head from an outside perspective in accordance with an illustrative embodiment. As shown in FIG. 10A, an illustrative ultrasound probe 1000 includes an electronics chamber 1005 and a probe head 1010. FIG. 10B shows the probe head 1010 from a front perspective. FIG. 10C shows the ultrasound probe 1000 with the electronics chamber 1005 detached from the probe head 1010. FIG. 10D shows the ultrasound probe 1000 with the electronics chamber 1005 detached from the probe head 1010 from a top perspective and FIG. 10E shows the ultrasound probe 1000 with the electronics chamber 1005 detached from the probe head 1010 from a side perspective.

In an illustrative embodiment, any of the operations described herein can be implemented at least in part as computer-readable instructions stored on a computer-readable memory. Upon execution of the computer-readable instructions by a processor, the computer-readable instructions can cause a node to perform the operations.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A device for creating images via ultrasonic pulses, the device comprising:
an electronics chamber comprising a motor with an output shaft; and
a probe head attached to the electronics chamber, wherein the probe head includes a liquid-filled chamber that comprises:
an ultrasonic transducer configured to transmit and receive ultrasonic pulses; and
a mirror configured to reflect the ultrasonic pulses, wherein the mirror is configured to rotate;
wherein the output shaft of the motor and the mirror are rotationally coupled.

2. The device of claim 1, wherein the electronics chamber further comprises a first magnet mounted to the output shaft, wherein the probe head further comprises a second magnet rotationally connected to the mirror, and wherein the output shaft of the motor and the mirror are rotationally coupled via the first magnet and the second magnet.

3. The device of claim 2, wherein the first magnet magnetically interacts with the second magnet and causes the second magnet to orient magnetic poles of the second magnet opposite an orientation of the magnetic poles of the first magnet

4. The device of claim 2, wherein the first magnet and the second magnet each comprise two magnets.

5. The device of claim 2, further comprising a first set of ball bearings around the output shaft of the motor and a second set of ball bearings around a shaft mounted to the second magnet.

6. The device of claim 5, wherein the electronics chamber further comprises:
an electronics chamber wall between the first magnet and the probe head, and
a spacer located between the first set of ball bearings and the electronics chamber wall, and
wherein the probe head further comprises:
a probe head wall between the electronics chamber and the second magnet, and
a spacer located between the second set of ball bearings and the probe head wall.

7. The device of claim 6, wherein the first magnet is between the electronics chamber wall and the first set of ball bearings, and wherein the second magnet is between the probe head wall and the second set of ball bearings.

8. The device of claim 2, wherein the probe head further comprises a mirror shaft that is mounted to the second magnet and the mirror.

9. The device of claim 2, wherein the probe head further comprises:
a first shaft mounted to the second magnet; and
a second shaft mounted to the mirror,
wherein the first shaft and the second shaft are perpendicular to one another.

10. The device of claim 9, wherein the first shaft and the second shaft are mechanically connected via bevel gears.

11. The device of claim 1, wherein the electronics chamber and the probe head are detachable from one another.

12. The device of claim 11, wherein the electronics chamber comprises one of a tongue or a groove and the probe head comprises of the other of the groove or the tongue, and wherein each of the tongue and the groove of the electronics chamber and the probe head are configured to align the electronics chamber and the probe head.

13. The device of claim 12, wherein the electronics chamber and the probe head are aligned to facilitate a magnetic interaction between a first magnet mounted to the output shaft and a second magnet mechanically coupled to the mirror.

14. The device of claim 1, wherein the output shaft of the motor is not mechanically coupled to the mirror.

15. The device of claim 1, wherein the probe head further comprises a lens configured to direct the ultrasonic pulses.

16. The device of claim 15, wherein the mirror reflects the ultrasonic pulses while rotating, and wherein received ultrasonic pulses are used to determine an ultrasonic scan of material adjacent to the lens.

17. The device of claim 1, wherein the mirror is configured to rotate at a rotational speed of the motor.

18. The device of claim 2, wherein an axis of rotation of the first magnet is perpendicular to an axis of rotation of the second magnet.

19. The device of claim 1, wherein the electronics chamber further comprises a battery and electrical circuitry, wherein the electrical circuitry is electrically connected to the ultrasonic transducer.

20. The device of claim 1, wherein the motor is not surrounded by a liquid.
